(19) United States
(12) Patent Application Publication
(10) Pub. No.: US 2022/0301659 A1
(43) Pub. Date: Sep. 22, 2022

(54) FULL-SPECTRUM PREDICTION OF MOLECULES TANDEM MASS SPECTRA USING DEEP NEURAL NETWORK

(71) Applicant: The Trustees of Indiana University, Bloomington, IN (US)

(72) Inventors: Haixu Tang, Bloominton, IN (US); Kaiyuan Liu, Bloomington, IN (US); Yuzhen Ye, Bloomington, IN (US)

(21) Appl. No.: 17/616,072

(22) PCT Filed: Jun. 3, 2020
(86) PCT No.: PCT/US20/35949

§ 371 Date: Dec. 2, 2021

Related U.S. Application Data
(60) Provisional application No. 62/856,948, filed on Jun. 4, 2019.

(51) Int. Cl. G16B 40/10; G16B 40/20; G16B 15/20; G06N 3/12

(52) U.S. Cl. CPC G16B 40/10; G16B 40/20; G16B 15/20; G06N 3/126

(57) ABSTRACT

Method and system for predicting a complete tandem mass spectrum of a molecule are disclosed. For example, the method includes training a prediction model using a dataset with features that incorporate at least one physiochemical feature derived from one or more peptide sequences and predicting complete tandem mass spectra of a molecule using the prediction model, the complete tandem mass spectra including backbone fragment ions and non-backbone fragment ions.

### RELATED APPLICATIONS

The present application claims the benefit of U.S. Provisional Patent Application No. 62/856,948, entitled “FULL-SPECTRUM PREDICTION OF MOLECULES TANDEM MASS SPECTRA USING DEEP NEURAL NETWORK,” and filed Jun. 4, 2019, the entire disclosure of which is hereby expressly incorporated by reference herein in its entirety.

### GOVERNMENT SUPPORT CLAUSE

This invention was made with government support under AI108888 awarded by National Institutes of Health. The government has certain rights in the invention.

### FIELD OF THE DISCLOSURE

The present disclosure generally relates to mass spectrometry (MS) technology, and more particularly to methods and systems for predicting tandem mass (MS/MS) spectra of peptides.

### BACKGROUND OF THE DISCLOSURE

The mass spectrometry (MS) technology, in particular, the liquid chromatography coupled tandem mass spectrometry (LC-MS/MS), has evolved rapidly in the past decade, with improved throughput and sensitivity. Many large-scale proteomic and metabolomic projects have been launched for various diseases, including cardiovascular diseases, diabetes, and cancer. These studies often involved hundreds to thousands of clinical samples, generating massive MS/MS datasets, as in the case of other sequencing-based ‘omics’ fields like genomics and transcriptomics. To make the maximum use of such data, a community effort represented by the ProteomeXchange consortium (current members including the PRIDE Archive, PeptideAtlas, MassIVE, and jPOST) was launched for public repository of proteomics data. As a result, the number of publicly accessible proteomic MS/MS datasets has grown exponentially in the past few years. Publicly available MS/MS datasets may be used for predicting peptide tandem mass (MS/MS) spectra. The ability to predict MS/MS spectra of peptides may enhance the understanding of mass spectrometry and improve peptide identification in proteomics.

### BRIEF SUMMARY OF THE DISCLOSURE

The present embodiments relate to computer systems and methods that may improve predicting MS/MS spectra from a peptide sequence.

In one aspect, a method for predicting complete tandem mass spectra of a molecule is provided. The method includes training a prediction model using a dataset with features that incorporate at least one physiochemical feature derived from one or more peptide sequences and predicting complete tandem mass spectra of a molecule using the prediction model, the complete tandem mass spectra including backbone fragment ions and non-backbone fragment ions.

In some embodiments, the dataset may include a plurality of fragment peaks of the one or more peptide sequences without fragment ion annotations or fragmentation rules.

In some embodiments, training the prediction model using the dataset may include inputting the at least one physiochemical feature derived from one or more peptide sequences and learning physiochemical rules governing peptide fragmentation to predict fragmentation rules.

In some embodiments, predicting MS/MS spectra of a molecule may include predicting one or more occurrences and intensities of backbone fragment ions and/or non-backbone fragment ions.

In some embodiments, predicting the complete tandem mass spectra of the molecule may include determining an intensity vector for each peak of experimental spectra and predicted spectra, normalizing intensity vectors to avoid being dominated by one or more intensive peaks, determining a cosine similarity of the normalized intensity vectors between experimental spectra and predicted spectra, and comparing the cosine similarity.

In some embodiments, the molecule may be selected from the group consisting of a peptide, a metabolite, a lipid, and a glycan.

In some embodiments, the molecule may be a peptide.

In some embodiments, the peptide may be a modified peptide.

In some embodiments, the dataset may include a plurality of fragment peaks from at least one of high-energy collisional dissociation (HCD) spectra, electron transfer dissociation (ETD) spectra, and/or collision-induced dissociation (CID) spectra.

In another aspect, a computing device for predicting complete tandem mass spectra of a molecule is provided. The computing device includes a processor and a memory having a plurality of instructions stored thereon that, when executed by the processor, causes the computing device to: train a prediction model using a dataset with features that incorporate at least one physiochemical feature derived from one or more peptide sequences and predict complete tandem mass spectra of a molecule using the prediction model, the complete tandem mass spectra including backbone fragment ions and non-backbone fragment ions.

In some embodiments, the dataset may include a plurality of fragment peaks of the one or more peptide sequences without fragment ion annotations or fragmentation rules.

In some embodiments, to train the prediction model using the dataset may include to input the at least one physiochemical feature derived from one or more peptide sequences and learn physiochemical rules governing peptide fragmentation to predict fragmentation rules.

In some embodiments, to predict MS/MS spectra of a molecule may include to predict one or more occurrences and intensities of backbone fragment ions and/or non-backbone fragment ions.

In some embodiments, to predict the complete tandem mass spectra of the molecule may include to determine an intensity vector for each peak of experimental spectra and predicted spectra, normalize intensity vectors to avoid being dominated by one or more intensive peaks, determine a cosine similarity of the normalized intensity vectors between experimental spectra and predicted spectra, and compare the cosine similarity.

In some embodiments, the molecule may be selected from the group consisting of a peptide, a metabolite, a lipid, and a glycan.

In some embodiments, the molecule may be a peptide.

In some embodiments, the peptide may be a modified peptide.

In some embodiments, the dataset may include a plurality of fragment peaks from at least one of high-energy collisional dissociation (HCD) spectra, electron transfer dissociation (ETD) spectra, and/or collision-induced dissociation (CID) spectra.

In other aspect, a non-transitory computer-readable medium storing instructions for a status of a mobile device of a user is provided. The instructions when executed by one or more processors of a computing device, cause the computing device to train a prediction model using a dataset with features that incorporate at least one physiochemical feature derived from one or more peptide sequences and predict complete tandem mass spectra of a molecule using the prediction model, the complete tandem mass spectra including backbone fragment ions and non-backbone fragment ions.

In some embodiments, the dataset may include a plurality of fragment peaks of the one or more peptide sequences without fragment ion annotations or fragmentation rules.

In some embodiments, to train the prediction model using the dataset may include to input the at least one physiochemical feature derived from one or more peptide sequences and learn physiochemical rules governing peptide fragmentation to predict fragmentation rules.

In some embodiments, to predict MS/MS spectra of a molecule may include to predict one or more occurrences and intensities of backbone fragment ions and/or non-backbone fragment ions.

In some embodiments, to predict the complete tandem mass spectra of the molecule may include to determine an intensity vector for each peak of experimental spectra and predicted spectra, normalize intensity vectors to avoid being dominated by one or more intensive peaks, determine a cosine similarity of the normalized intensity vectors between experimental spectra and predicted spectra, and compare the cosine similarity.

In some embodiments, the molecule may be selected from the group consisting of a peptide, a metabolite, a lipid, and a glycan.

In some embodiments, the molecule may be a peptide.

In some embodiments, the peptide may be a modified peptide.

In some embodiments, the dataset may include a plurality of fragment peaks from at least one of high-energy collisional dissociation (HCD) spectra, electron transfer dissociation (ETD) spectra, and/or collision-induced dissociation (CID) spectra.

Embodiments of the invention include a method to predict a complete tandem mass spectrum of a molecule utilizing the step of: training a neural network algorithm using a data set with features that incorporate at least one physiochemical feature from at least one molecule. In one embodiment, the molecule is selected from the group consisting of a peptide, a metabolite, a lipid and a glycan. In another embodiment, the molecule is a peptide. In a further embodiment, the peptide is a modified peptide.

### BRIEF DESCRIPTION OF THE DRAWINGS

FIGS. 1A and 1B show a tensor presentation of the residual convolutional neural network.

FIGS. 2A and 2B show similarities between the experimental and predicted HCD spectra for +2 (FIG. 2A) and +3 (FIG. 2B) precursor peptides ions, in comparison with the similarities between spectra in replicated experiments.

FIGS. 3A and 3B illustrate predicted (top panel) versus experimental (bottom panel) spectra with charges of +2 (FIG. 3A) and +3 (FIG. 3B).

FIGS. 4A and 4B show an intensity composition of fragment ion types in experimental versus predicted spectra for +2 (FIG. 3A) and +3 (FIG. 3B) precursors.

FIG. 5 illustrates the similarities between the experimental and predicted HCD spectra of peptides with different lengths.

FIG. 6 demonstrates that the accuracy of predicted spectra is highly correlated with similarity between replicated spectra across experiments for the same peptides.

FIGS. 7A and 7B show that prediction accuracy for +2 spectra (FIG. 7A) and +3 spectra (FIG. 7B) (measured by the similarity between the predicted and experimental spectra; y-axis) increases with more training data (x-axis).

FIG. 8 shows the M/z shifts distribution of replicates.

FIG. 9 illustrates non-matches distribution for raw intensities.

FIG. 10 shows the average similarities between replicated HCD spectra (of all charges) of peptides with different lengths.

FIG. 11 illustrates the similarities between the experimental and predicted HCD spectra on the spectra with different number of fragment ions.

FIG. 12 shows a core architecture of the residual convolutional neural network (CNN) model for spectrum prediction.

FIG. 13 illustrates that a prediction accuracy (measured by the similarity between the predicted and experimental spectra on testing data; y-axis) increases with more training data (x-axis).

FIG. 14 illustrates a multitask learning model for joint training of HCD and ETD Spectra with all charge states (1+, 2+, 3+ and 4+).

FIGS. 15A and 15B illustrate similarities between experimental and predicted HCD spectra for 2+ (FIG. 15A) and 3+ (FIG. 15B) precursor peptides ions, in comparison with the similarities between spectra in replicated experiments and other approaches.

FIGS. 16A and 16B illustrate similarities on the b/y ion intensities between the experimental and predicted HCD spectra: results for charge 2+ (FIG. 16A) and results for charge 3+ (FIG. 16B).

FIGS. 17A and 17B illustrate predicted (bottom half) HCD spectra versus experimental (top half) HCD spectra of charges 2+ (FIG. 17A) and 3+ (FIG. 17B). Note that the intensities are transformed by the square root function.

FIGS. 18A and 18B illustrate similarities between the experimental and predicted 1+ (FIG. 18A) and 4+ (FIG. 18B) HCD spectra using a multitask learning (MTL) approach, in comparison with the similarities between spectra in replicated experiments and the direct prediction approach.

FIG. 19A-19C illustrate similarities between the experimental and predicted ETD spectra using MTL approach for 2+ (FIG. 19A), 3+ (FIG. 19B) and 4+ (FIG. 19C) precursor peptides ions, in comparison with the similarities between spectra in replicated experiments and the direct prediction approach.

FIG. 20 illustrates predicted (bottom half) ETD spectra versus experimental (top half) ETD spectra of charge 3+. Note that the intensities are transformed by the square root function.

FIGS. 21A and 22B illustrate similarity distribution of full spectrum or backbone-only spectrum with its replicates: similarity distribution of charge 2+ HCD spectra (FIG. 21A) and similarity distribution of charge 3+ HCD spectra (FIG. 21B).

FIGS. 22A-22D illustrate intensity composition of fragment ion types in experimental (FIGS. 22A and 22C) versus predicted (FIGS. 22B and 22D) HCD spectra for 2+ (FIGS. 22A and 22B) and 3+ (FIGS. 22C and 22D) precursor ions.

FIGS. 23A and 23B illustrate average intensities of different fragment ions in experimental (FIG. 23A) and predicted (FIG. 23B) ETD spectra of charges 1+ to 4+.

FIGS. 24A and 24B illustrate the accuracy of predicted spectra is highly correlated with similarity between replicated spectra across experiments for the same peptides: relationship of charge 2+ spectra (FIG. 24A) and relationship of charge 3+ spectra (FIG. 24B).

FIG. 25A illustrates the similarities between the experimental and predicted HCD spectra decrease with the increasing peptide length. The statistics were conducted over 10,000 HCD spectra of charge 2+.

FIG. 25B illustrates the similarities between replicated HCD spectra decrease with the increasing peptide length. The statistics ware conducted over 10,000 randomly sampled experimental HCD spectra of charge 2+.

FIGS. 26A and 26B illustrate the distribution of m/z shifts between replicated HCD spectra of charge 2+ (FIG. 26A) and 3+ (FIG. 26B). Both statistics were conducted over 10,000 HCD spectra of charge 2+ and charge 3+.

FIGS. 27A-27C illustrate the distributions of similarities between the replicated experimental spectra of the same peptides versus those between two distinct peptides with the same precursor mass when different normalization functions were applied to the intensities of fragment ions. The statistics ware conducted over 5,000 randomly sampled HCD spectra of charge 2+: Original intensities (FIG. 27A), Intensities transformed by Log (FIG. 27B), and Intensities transformed by square root (FIG. 27C).

FIGS. 28A-28C illustrate the decreasing of the losses (y-axis) on the training and testing data along with the training history (x-axis: number of epochs). FIG. 28A illustrates a total loss; the training and testing losses are close, indicating the model does not over-fit to the training data. FIG. 28B illustrates that a loss of the spectra prediction task. FIG. 28C illustrates other losses of auxiliary tasks, which quickly drops to nearly zero as expected.

Corresponding reference characters indicate corresponding parts throughout the several views. Although the drawings represent embodiments of the present disclosure, the drawings are not necessarily to scale, and certain features may be exaggerated in order to better illustrate and explain the present disclosure. The exemplification set out herein illustrates an embodiment of the disclosure, in one form, and such exemplifications are not to be construed as limiting the scope of the disclosure in any manner.

### DETAILED DESCRIPTION

For the purposes of promoting an understanding of the principles of the present disclosure, reference is now made to the embodiments illustrated in the drawings, which are described below. The exemplary embodiments disclosed herein are not intended to be exhaustive or to limit the disclosure to the precise form disclosed in the following detailed description. Rather, these exemplary embodiments were chosen and described so that others skilled in the art may utilize their teachings. One of ordinary skill in the art will realize that the embodiments provided can be implemented in hardware, software, firmware, and/or a combination thereof. Programming code according to the embodiments can be implemented in any viable programming language or a combination of a high-level programming language and a lower level programming language.

Different approaches have been proposed for the prediction of peptide MS/MS spectra. For example, the MassAnalyzer explicitly models a chemical process of peptide fragmentation with parameters optimized using annotated MS/MS spectra. Other models like SeQuence IDentfication (SQID) tried to make predictions based on statistical results of peak intensities from annotated MS/MS spectra. In contrast, the machine learning (ML) approaches have been proposed to predict MS/MS spectra from peptide sequences. The ML models are designed to be trained using annotated peptide spectra and predict a probability of observing each fragment ion (e.g., b-, y-ions and neutral loss ions) in an experimental spectrum.

Since the development of these prediction algorithms, a significant advancement has been made in mass spectrometry techniques. It has been shown that the reproducibility of peptide MS/MS spectra resulting from higher-energy collisional dissociation (HCD) are generally higher than the collision-induced dissociation (CID) spectra used in the training and testing by the early rule-based prediction algorithms. On the other hand, because of the availability of more identified peptide spectra and the rapid advance of ML algorithms, it is feasible to train complex deep learning models that require a large training set to automatically learn physiochemical rules governing peptide fragmentation, and thus make more accurate predictions than the relatively simple neural networks, as demonstrated in a recently developed peptide spectra predictor pDeep, DeepMass, and Prosit. However, although pDeep explicitly models the intensity dependencies among b/y ions (e.g., those between bᵢ and yₙ₋ᵢ, and between bᵢ and bᵢ₋₁, etc.) using a recurrent neural network (RNN), pDeep and other deep learning-based spectra prediction tools (e.g., PRISM/DeepMass) followed the same framework of predicting the intensity of expected fragment ions (e.g., b/y ions) only, which are derived based on rational fragmentation rules (e.g., the peptide bond cleavage in HCD/CID spectra). It should be appreciated that these approaches may be limited to predict intensities of expected fragment ion types (i.e., a/b/c/x/y/z ions and their neutral loss derivatives, referred to as backbone ions). As such, these approaches are referred to as the backbone-only predictors or rule-based spectrum predictors. In practice, the backbone ions account for less than 70% of total ion intensities in HCD spectra, indicating many intense ions are ignored by these predictors.

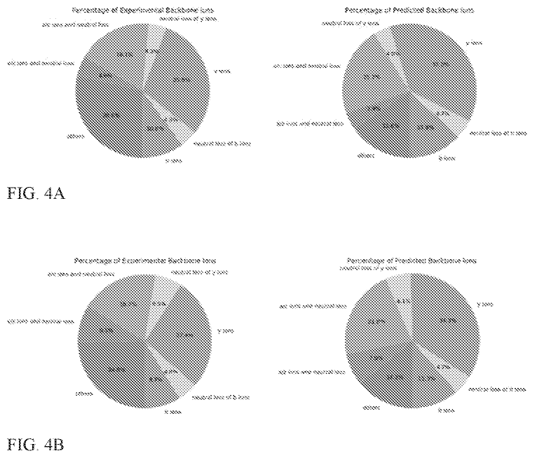

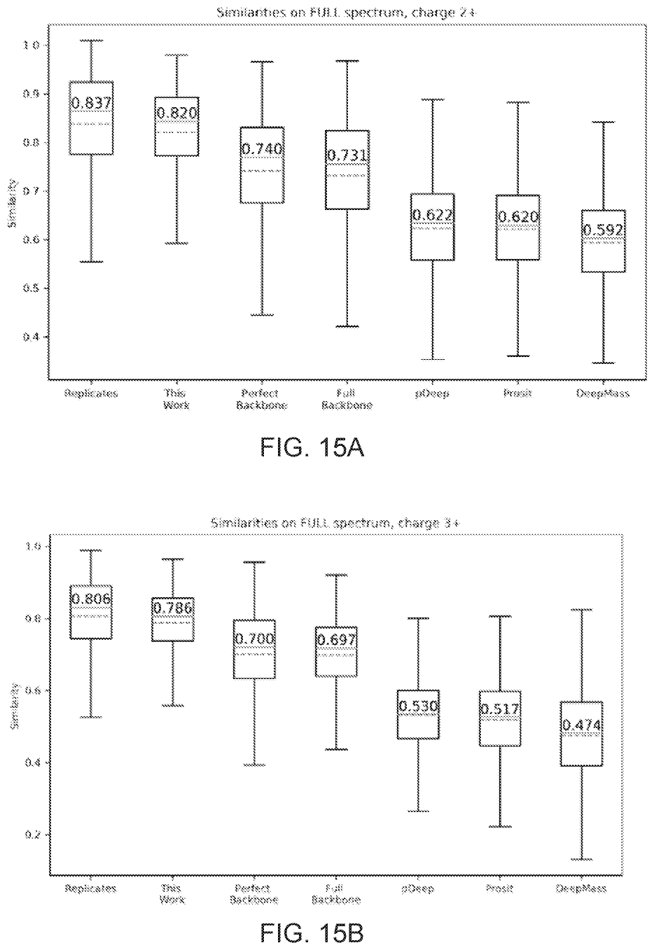

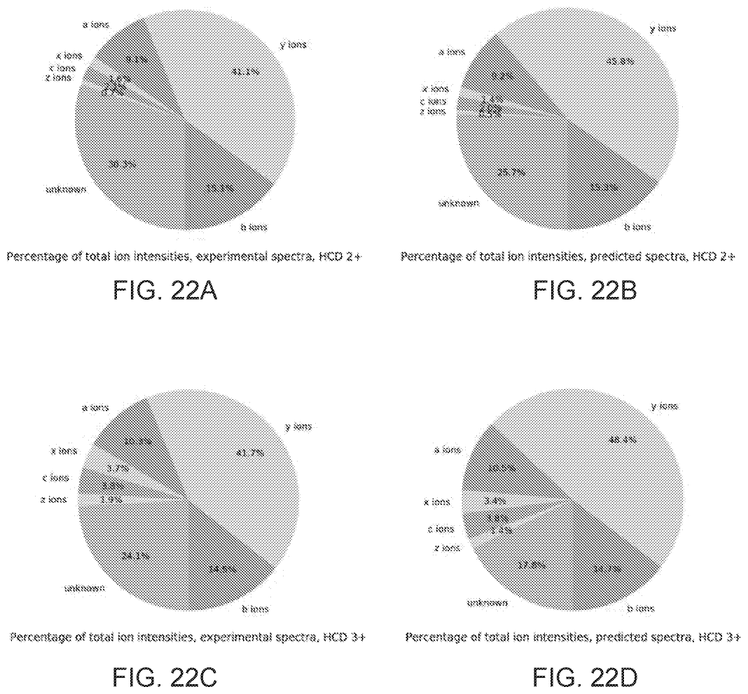

In contrast, this application discloses a deep learning approach that predicts a complete MS/MS spectra, both backbone and non-backbone ions, directly from peptide sequences. For example, as described further below, a substantial fraction (˜30%) of ions in HCD spectra cannot be annotated as a/b/c/x/y/z ions or their neutral loss derivatives (i.e., backbone ions). See FIGS. 4A, 4B, and 22A-22D. As a result, even a method that can perfectly predict the intensities of all backbone ions is likely to lack around 30% of peaks. Even if a sub-spectrum containing only backbone ions is extracted in the spectrum in attempt to generate hypothetical perfect predictions, its average similarity with its full spectrum replicates is still far from that between the replicated full spectra. See FIGS. 15A, and 15B. In other words, even if a hypothetical algorithm can predict the exact intensities of all backbone ions, the similarity between the predicted and experimental spectra is not sufficiently high. As such, the prediction of full spectrum is employed to improve the overall similarity between replicated peptide spectra. Notably, the mechanic explanation of these non-backbone fragment ions are lacking, and thus it is non-trivial to provide fragmentation rules to guide machine learning algorithms to learn the intensities of these ions.

On the other hand, with a sufficient amount of training, deep learning models may automatically discover complex rules and patterns by itself (e.g., the patterns of natural images). The illustrative systems and methods utilize the capability of deep learning models to self-learn and discover the fragmentation rules from a large number of training samples without fragment ion annotations and simultaneously predict the occurrences and intensities of fragment ions. It should be appreciated that the illustrative systems and methods (i) do not make assumptions or expectations on which kind of ions to predict and (ii) provide no annotations of fragment ion or fragmentation rules to ML models. Instead, the illustrative systems and methods are configured to predict intensities at all possible m/z values and, thus, not limited to given ions types. It should also be appreciated that the illustrative systems and methods may also be applied to the prediction of MS/MS spectra of other molecules, e.g., metabolites, lipids, and glycans, and the prediction of peptide MS/MS spectra using other fragmentation methods, e.g., the high energy HCD or electron transfer/high-energy collision dissociation (EThcD), in which the fragmentation rules are more complex and less understood.

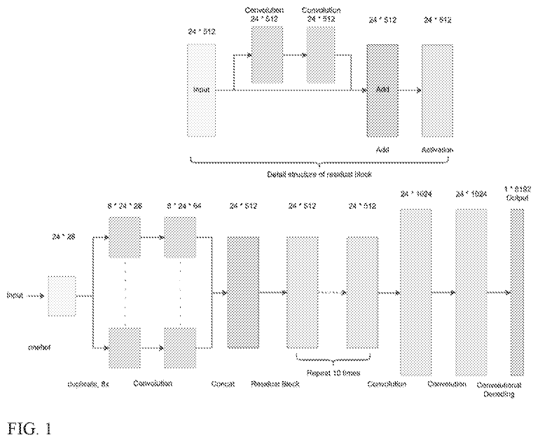

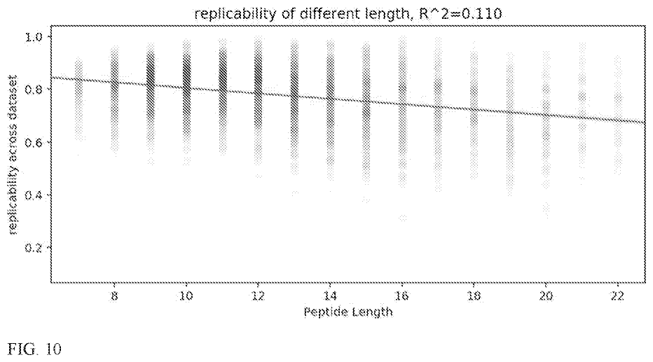

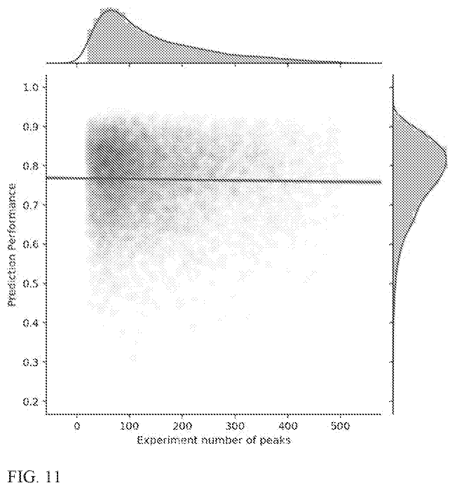

Results for FIGS. 1-11

Deep learning model. A generalized sequence-to-sequence (Seq2Seq) model (also referred to as the prediction model in this application) was developed based on the structure of residual convolutional neural network (CNN) for predicting full peptide MS/MS spectra from peptide sequences. As depicted in FIG. 1, for the encoder part of the network, the peptide sequence was first embedded into a one-hot encoded vector encoding, with the amino acid masses and other necessary meta-information as well. The embedded representation will then be fed into 16 separate convolutional layers of different kernel sizes (from 2 to 17). The step is designed to capture the correlations among subsequences of encoded peptide. Afterwards, the convolution results are concatenated into a single tensor that the information of every subsequences of different lengths are combined. This tensor will be passed into 10 (or more) consequential residual blocks. Because residual block can prevent gradient vanishing during training using a gradient descent method, it allows more hidden layers to be stacked. The result output of 512 channels will be regard as the representation of feature tensor for subsequent decoding operations.

The decoder part of the CNN takes the feature tensor as an input and uses additional three convolutional residual blocks to extend the tensor to 1024 channels. The design of these blocks follows that of the SENe. A final convolutional layer will decode the tensor into an 8000 dimension (or higher) vectorized presentation of the MS/MS spectrum, depending on the desirable mass resolution. The default 8000 dimension in the illustrative model corresponds to the mass resolution of about 0.2 Da. It should be appreciated that in some embodiments, the accuracy of predicted spectra may not be improved and the training may take much longer to predict higher dimension output vectors (i.e., with higher mass resolution, e.g., 0.05 Da, corresponding to the output vector of about 32,000 dimensions). In the illustrative embodiment, the vectorized prediction is further refined to remove dubious peaks (mostly noisy peaks) before converted into the final spectrum prediction.

It should be noted that commonly used pooling layers in CNN were not incorporated in the illustrative model architecture, which, along with the residual neural network structure, is critical for the good performance of the illustrative model according to the experiments described herein. Additionally, the illustrative model was used to simultaneously predict the precursor ion mass of the input peptide. FIG. 1 shows the tensor presentation of the residual convolutional neural network. The entire residual CNN contains about 18 millions of parameters and occupies the space of 70 M.

Training models for predicting doubly and triply charged HCD spectra. The deep learning model was implemented using the Tensorflow framework, and the models were first trained for predicting doubly (+2) and triply (+3) HCD spectra of peptides because of the massive number of such spectra are publicly available at MS data repositories. In the illustrative embodiment, the spectral libraries, including the NIST HCD library, the NIST Synthetic HCD library, the Human HCD library from MassIVE, and the synthetic HCD library from ProteomeTools, were used. In total, around 1.5 million +2 spectra and 1 million +3 spectra were used for the training process. About 25 thousand +2 and 20 thousand +3 spectra were held out for testing purpose, respectively, from the peptides that do not overlap with the training samples. Detail amounts about these datasets are listed in Table 1. Specifically, the NIST HCD library was used for training only, because it is a relatively old dataset with comparably lower data quality. Meanwhile, testing PSMs are only selected form the NIST Synthetic, the ProteomeTools synthetic library, and the MassIVE Human HCD library, while the remaining data were used in the training process. In addition, the NIST Hamster dataset was used only for testing purpose to ensure that illustrative prediction model can be generalized to peptide sequences that are not similar with the training sequence. In the illustrative embodiment, samples with observed peaks less than 20 or more than 500 (over fragmented) were ignored. Additionally, the peptide length was limited to 22 and precursor mass up to 2000, as those spectra are rare in practice and also rare in the dataset.

It should be noted that the types of instruments used to acquire these HCD spectra are not distinguished because the HCD spectra generated by different instruments (e.g., Orbitrap, Fusion, and Q Exactive) are highly similar. However, the instrument setting may affect the similarity among replicated spectra, as presented below. Also, as not all training samples contain information of normalized collision energy (NCE), all unlabeled samples were assumed by NCE of 20%. Unexpectedly and fortunately, it is determined that the impact of NCE were relatively small.

Model performance on doubly and triply charged HCD spectra. To evaluate the accuracy of the predicted MS/MS spectra, the cosine similarities were computed between the experimental and the predicted spectra by the illustrative prediction model on the testing data with 25 K +2 spectra and 20 K +3 spectra, as shown in Table 1. For example, as shown in Table 2, the similarities were computed between the replicated HCD spectra of the same peptides in different libraries (experiments) as well as the similarities between the experimental and the predicted spectra by pDeep using the rule-based approach. Furthermore, for each testing case, a perfect b/y spectrum consisting of only backbone ions (including b/y, c/z, a/x, and their derivative neural losses peaks) were generated in the experimental spectrum, and the other ions were removed, which represents the best case that any rule-based spectrum prediction algorithm can achieve if it only predicts the intensities of backbone ions. Spectrum similarities were also computed using other measures instead of cosine similarity (e.g., Pearson correlation, etc.) and with different type of intensity normalization method (e.g., logarithm normalization). The general trends of the prediction performance are similar as the results presented below.

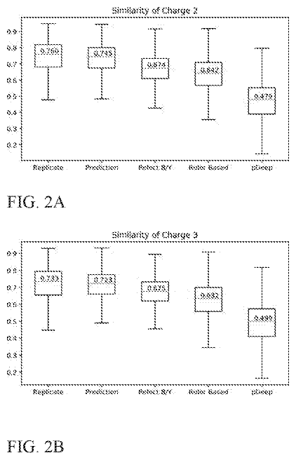

As shown in FIG. 2, the spectra predicted by the illustrative algorithm are highly similar with the experimental spectra, with the average full-spectrum cosine similarities of 0.755 (±0.088) and 0.728 (±0.089) for +2 and +3 HCD spectra, respectively. In contrast, the average full-spectrum cosine similarities between the replicated spectra of the same peptides are 0.776 (±0.11) and 0.761 (±0.11) for +2 and +3 spectra, respectively, implying that the illustrative models approach the optimal prediction accuracy. In contrast, even a perfect rule-based prediction algorithm can only achieve the average cosine similarities around 0.665 (±0.09) and 0.675 (±0.104) for +2 and +3 spectra, respectively. However, because it is impractical to achieve the perfect prediction in practice, the average cosine similarities achieved by the rule-based prediction were obtained by the refined implementation of pDeep and are around 0.626 (±0.08) and 0.631(±0.09) for +2 and +3 spectra, respectively. Additionally, the original pDeep software, which does not consider all possible backbone ions, and can only achieve the average cosine similarities of 0.471 (±0.06) and 0.489 (±0.07) for +2 and +3 spectra, respectively.

The illustrative prediction model predicts almost perfect intensities of backbone ions, with average cosine similarities of 0.91 (±0.07) and 0.87 (±0.08) on these ions' intensities in the +2 and +3 spectra, respectively. These results showed that the illustrative deep learning model can discover the fragmentation rules (e.g., the m/z of all fragment ions and their intensities) from massive MS/MS spectra, consistent with the recent successes of deep learning algorithms on learning hidden rules and patterns.

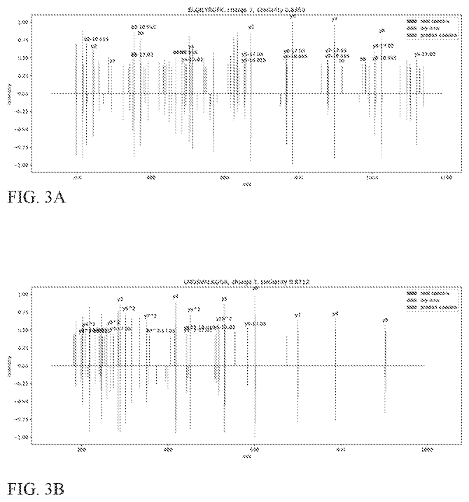

As shown in FIGS. 3A and 3B, the illustrative prediction algorithm can output full MS/MS spectra including non-backbone ions. It should be noted that the backbone ions peaks are illustrated in the lower half of each figure between −1.00-0 intensity, while the non-backbone ion peaks are illustrated in the upper half of each figure between 0-1.00 intensity. Firstly, it can cover almost all backbone ion peaks, indicating that the illustrative prediction model is robust and covers most desirable peaks. Additionally, the illustrative prediction algorithm successfully predicted most intensive non-backbone ion peaks observed in the experimental spectra, showing that these peaks represent fragmentation signals (even though the mechanism remains unknown) that can be captured by the learning algorithm. Finally, the prediction algorithm is more likely to miss some peaks than predicting non-observed peaks, which indicates that the learning algorithm tends to ignore these peaks (i.e. treating them as random noise) until it confirms that they are real signals. Overall, the illustrative prediction demonstrates a clear improvement over rule-based spectrum prediction algorithms.

Referring now to FIGS. 4A and 4B, the composition of fragment ions in the predicted versus experimental MS/MS spectra were compared by depicting the average percentage of total intensities for different types of fragment ions in the predicted and experimental spectra of testing cases. The composition of fragment ions in the predicted spectra by the illustrative method is similar to that in the experimental spectra, confirming that the illustrative prediction algorithm reliably predicts non-backbone ions. Notably, the overall backbone ion intensities in the predicted spectra are higher those in the experimental spectra, probably due to the presence of non-replicable noise peaks in the experimental spectra that are typically not predictable by the prediction algorithm. In the experimental HCD spectra, about 30% peak intensities are contributed by non-backbone (other) ions; in comparison, in the predicted spectra obtained by the illustrative method, these ions contribute about 15% peak intensities, which is smaller but still substantial. These predicted ions significantly boosted the similarity between the predicted and experimental spectra (FIG. 4). On the other hand, if the percentage of different type of backbone ions were to be plotted, the distribution is almost identical in predicted versus experimental spectra. For example, y-ions are most intensive followed by b-ions in both the predicted and experimental HCD spectra.

TABLE 1
Training and testing datasets.

| | NIST | NIST | ProteomeTools | | NIST |
|---|---|---|---|---|---|
| Datasets | HCD | Synthetic | Synthetic | MassIVE | Hamster |
| Charge 2 | 604536 | 344007 | 146555 | 598617 | 8947 |
| Charge 3 | 303532 | 193722 | 94105 | 491532 | 37392 |

TABLE 2
Average spectra similarities on peptide ions of different charges

| Similarity | Charge 2 | Charge 3 |
|---|---|---|
| Replicated spectra | 0.776 | 0.761 |
| Full-spectrum prediction | 0.755 | 0.728 |
| Perfect rule-based prediction | 0.665 | 0.675 |
| Refined pDeep | 0.626 | 0.631 |
| pDeep | 0.471 | 0.489 |

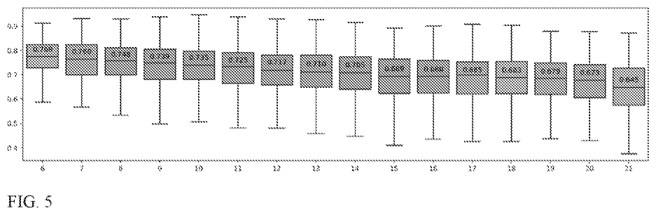

Variation of prediction accuracy. The prediction accuracy of the illustrative prediction model may vary depending on peptide lengths and replicability of the MS/MS spectra. As shown in FIG. 5, the prediction accuracy is relatively high for short peptides and gradually decreases as the length of peptides increases, especially for those peptides longer than 14 residues. This may be due to (1) intuitively, the spectra of long peptides may exhibit complex fragmentation patterns, and thus the prediction of long peptides are more challenging; (2) the training dataset contains fewer samples of longer peptides, which makes it difficult for the prediction model to learn fragmentation rules and patterns for these peptides, and (3) in fact, the similarities between replicated experimental HCD spectra also decrease as the length of peptides increases, which indicates that the signal to noise ratio may be reduced in the spectra of relatively longer peptides. In other words, more training samples with longer peptides may be used to train the prediction model to improve the prediction accuracy of the longer peptides.

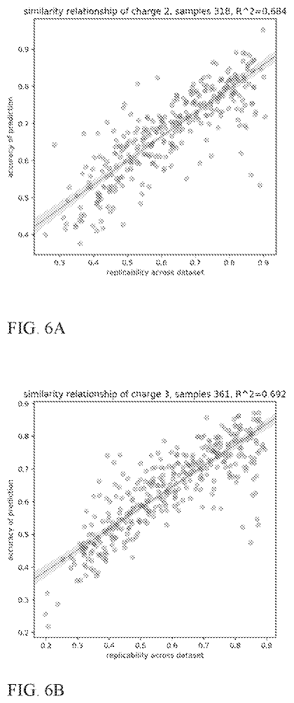

It was noted in FIG. 2 that the replicated spectra of some peptides exhibit relatively low similarity. An experiment was conducted to determine whether the prediction accuracy of those peptides' spectra are also relatively low. Indeed, as shown in FIGS. 6A and 6B, the similarity between replicated HCD spectra were highly correlated with the similarity between the experimental and predicted spectra of the same peptide. This indicates that the illustrative prediction deep learning model performs well on the highly replicable peptide spectra. On the other hand, the prediction accuracy was not affected by the complexity of experimental HCD spectra (e.g., measured by the number of fragment ions in HCD spectra. These results indicate that the predicted spectra are useful to validate confident peptide identifications, which are likely to be highly replicable.

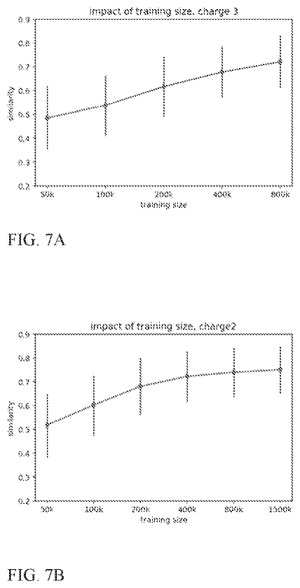

Power of massive training data. As predicted above, a total of 2.5 million training samples (including 1.5 million of +2 and 1 million of +3 spectra) were used to train the illustrative prediction model for the prediction of +2 and +3 spectra. FIGS. 7A and 7B show the power of massive training data: the prediction accuracy increases significantly with more spectra are employed as training samples. The trend for accuracy improvement gradually saturates when more than 1 million of training samples were used, which may indicate that the prediction accuracy of the prediction model may approach no more than 3% from an optimal predictor.

Learning of singly and quaternarily spectra. The MS/MS spectra of the same peptide of different charges may be drastically different. The training of the singly and quaternarily spectra may be, however, challenging because of the lack of the training data. As such, in some embodiments, representations of peptide learned from +2 and +3 peptide spectra, which has massive input training data, may be utilized to predict the +1 and +4 peptide spectra. To do so, a versatile prediction model that can simultaneously predict the spectra of multiple charges for the same input peptides may be generated. Such prediction model may not only save the efforts of building different models for predicting spectra of different charges, but also improve the representation learning of peptides by utilizing training samples from different charges.

Prediction of ETD Spectra. One of the challenges of predicting Electron-Transfer Dissociation (ETD) spectra is that there are much fewer reliable ETD datasets, around 200,000 samples, which is nearly 10 percent compared to HCD datasets. As such, a model that is directly trained by the fewer samples may not be reliable. Tentative training gave prediction similarity no more than 0.5, far from experiment replicates of similarity around 0.76.

In the illustrative embodiment, an ETD model was trained from pretrained HCD models. However, due to the phenomena of catastrophe forgetting, the final model may no longer be used to predict HCD peptides. The preliminary experiment of this approach gives a similarity of around 0.65 (±0.112).

Methods for FIGS. 1-11:

Data Preprocessing. It is natural to represent a spectrum as a 1-D vector. To do so, the m/z ranges are divided into many bins by a given bin width and the intensity is added with a bin as its value.

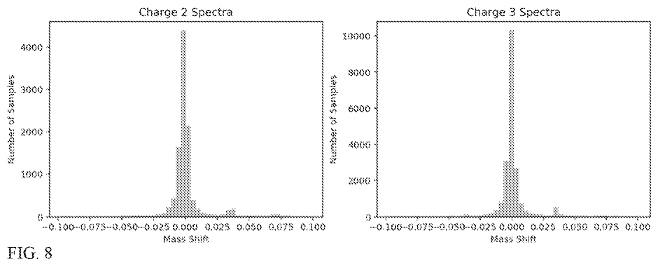

A good bin width is determined by the precision of spectra. Generally, the precision should not exceed the theoretical precision of the instrument, and it should be realistic compared to the precision that could be archived by experiments replicates. As shown in FIG. 8, the natural shift range of M/z are ignitable with precision 0.05 Da, thus, in the illustrative embodiment, the precision was selected to be slightly larger, at 0.1 Da.

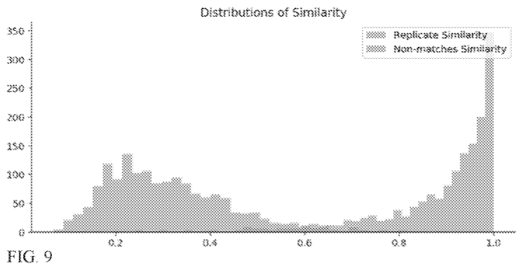

Subsequently, the similarity between a pair of spectra was evaluated as the cosine similarity of their corresponding vector representation. It should be appreciated that the similarity was not computed directly on raw intensities because the result will be dominated by several strongest peaks and thus gives inaccurate results. As shown in FIG. 9, the distribution of non-matches with raw intensities shared large overlapped ranges and thus requires a high threshold to yield results below some certain FDR. In other words, raw intensities will significantly decrease recall for a certain precision when calculating similarities.

Thus generally, the intensities were first normalized before the similarity was computed to avoid this problem. There are multiple ways to normalize the intensities (e.g., replace the raw intensity with its log or square root), In the illustrative embodiment, quadratic root was used as a normalize function for convincing, which gives similar result as log and needs no additional care of negative values. However, most non decreasing concave function may be used.

Implementation of Deep Neural Network (DNN). The deep neural network was implemented in Python using the Tensorflow framework with Keras front-end. The spectra prediction algorithm was also implemented as an independent software, which is released in open source and can also be accessed through a web service.

The training process takes ˜7×10⁻⁴ second per sample and spans 50 epochs on a single NVIDIA GTX1080ti GPU, while the prediction takes ˜10⁻³ second per peptide.

Using auxiliary tasks as focusing method could lead to better performance of deep learning models. For spectra prediction, the input precursor mass-to-charge (m/z) ratio is critical; thus, an auxiliary task was added to “predict” the precursor m/z, which enforce the deep learning model to fit the precursor. It should be appreciated that, in some embodiments, the precursor m/z may be predicted by computing from the input peptide sequence. Such prediction may work as a regulation for the deep learning model and may help to stabilize the training process.

A universal model for predicting HCD spectra of all changes. In some embodiments, a straightforward approach to build a universal model may be to use the mixed training dataset containing the HCD spectra of all charges while embedding the charges of each training sample as a separate input dimension. However, such approach cannot achieve satisfactory results because the training process may be dominated by of the most frequent +2 spectra. Indeed, the experiment results showed that the universal model trained in this way achieved the similar accuracy on the +2 HCD spectra as the model trained only on +2, while the performance of HCD spectra of the other charges (e.g., +3) is lower than the model trained only on the respective subset of spectra.

To address this issue, an auxiliary task approach was adopted to enforce a neural network to “predict” the precursor charges of the HCD spectra while predicting the spectra themselves. Similar to the auxiliary task of predicting precursor m/z, the auxiliary task of predicting the precursor changes may work as a regulation to stabilize the training of the deep learning model. The experimental results showed that a joint model training with auxiliary tasks gave similar or better results for the spectra of all charges.

Domain Adaptation. By the last approach, in some embodiments, the spectra of different fragmenting types may be considered as samples from different domains. By this assumption, domain adaptation methods that can erase the difference between ETD and HCD could help we find a universal model.

Discussion for FIGS. 1-11:

The illustrative prediction model was developed significantly different from those used by the existing rule-based spectrum predictors (e.g., pDeep and DeepMass): instead of predicting the intensity of each expected fragment ion (i.e., backbone ions in HCD spectra), the full MS/MS spectra was directly predicted, i.e., to predict both the m/z of the fragment ions and their intensities, not only on the expected backbone ions but on all ions. That means, the illustrative prediction model learns the complex chemical rules governing the fragmentation process of peptides without providing any prior knowledge, such as the frequent b/y and their derivative ions in HCD spectra (or the c/z ions in ETD spectra), or even the annotation of peptide-spectrum matches (PSMs), e.g., the ion species of observed peaks. As shown in the results and described further below, by exploiting the advantages of deep learning algorithms as well as the massive training sets of PSMs, these rules can be self-learned by deep learning methods. As a result, the non-backbone ions in HCD spectra, for which the fragmentation mechanisms may not be fully understood, can also be predicted, leading to much higher overall prediction accuracy, comparing to the existing rule-based methods that predict only backbone ion intensities.

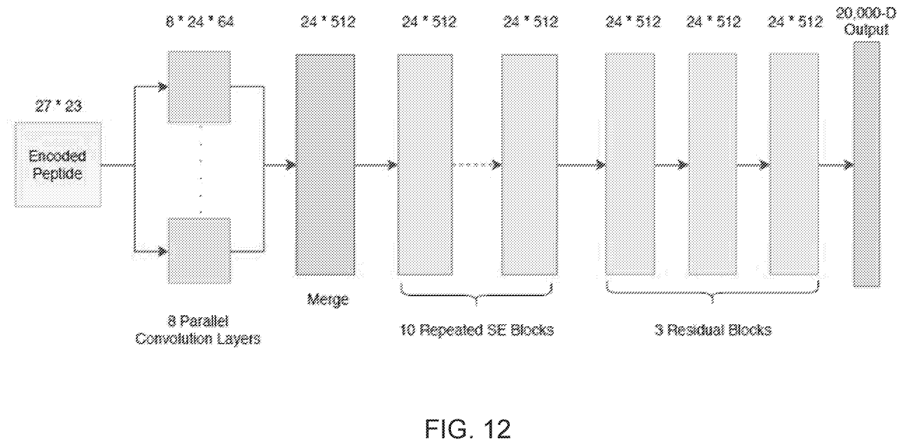

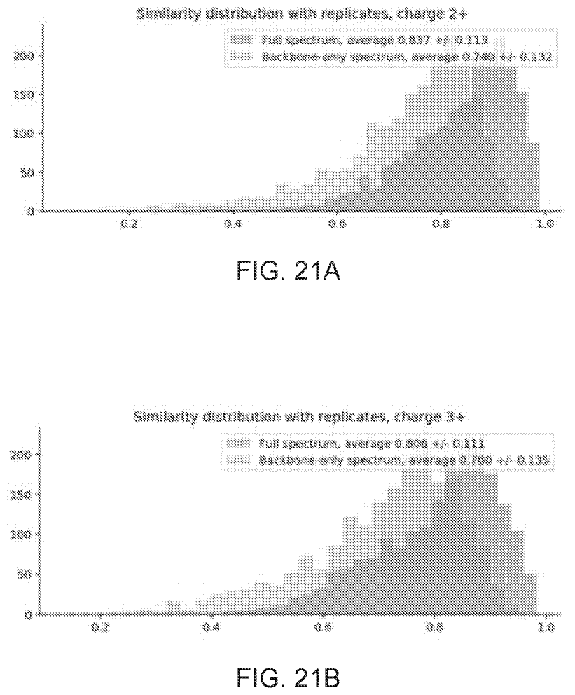

Methods for FIGS. 12-28:

Data and Evaluation Criteria. Identified HCD spectra were collected from spectral libraries including the NIST HCD library, the NIST Synthetic HCD library, the Human HCD library from MassIVE, and the synthetic HCD library from ProteomeTools. The sizes of these datasets are summarized in Table 3. In order to guarantee the quality of testing data, the NIST HCD library and the NIST synthetic HCD library, which are relatively old and with comparably lower data quality, were used for training only. Although testing samples were randomly selected from the original dataset, there are no overlaps between the training and testing peptides. As discussed further below, the training and testing datasets were further purified by removing under-fragmented PSMs, over-fragmented PSMs, (less than 1%), and PSMs with precursor mass difference more than 200 ppm. The complete training and testing datasets are available at the supplementary web site, http://www.predfull.com/datasets.

Data Selection. High-quality training data is critical for achieving good performance. As such, suspicious PSMs were filtered out to retain a more promising training set. In the illustrative embodiment and experiments, all spectra containing fewer than 20 peaks (i.e., under-fragmented) or more than 500 peaks (i.e., over-fragmented) were removed. Additionally, all PSMs with precursor mass mismatched more than 200 ppm were also removed. PSMs with peptide length greater than 25 or precursor mass greater than 2000 m/z were also excluded, as those spectra are relatively rare (e.g., less than 4 percent in our collected HCD spectra dataset).

TABLE 3
The total numbers of spectra in spectra libraries used for training and testing the
spectra prediction models for HCD and ETD spectra. The number in each cell means the
size of training data (including about 10% of validation data, used for choosing hyper-
parameters), while the numbers of testing samples are shown in the parentheses.

| | | | NIST | | | | | | |
|---|---|---|---|---|---|---|---|---|---|
| Type | Charge | NIST HCD | Synthetic | MassIVE | | ProteomeTools | | Total | |
| HCD | 1+ | 10,392 | 29 | 6,349 | (1,262) | 0 | | 16,770 | (1,262) |
| | 2+ | 536,701 | 320,062 | 512,105 | (16,989) | 126,586 | (7,620) | 1,495,454 | (24,609) |
| | 3+ | 189,933 | 140,273 | 309,239 | (14,342) | 59,736 | (5,438) | 699,181 | (19,780 |
| | 4+ | 18,190 | 15,762 | 50,428 | (4,494) | 7,203 | (1,046) | 91,583 | (5,540) |

| | | | | | | | | |
|---|---|---|---|---|---|---|---|---|
| ETD | 2+ | 0 | 0 | 26,254 | (4,666) | 0 | 26,254 | (4,666) |
| | 3+ | 0 | 0 | 129,647 | (17,208) | 0 | 129,647 | (17,208) |
| | 4+ | 0 | 0 | 10,274 | (3,405) | 0 | 10,274 | (3,405) |

Data Pre-processing. For the learning purpose, an MS/MS spectrum was represented as a sparse one-dimensional (1-D) vector by binning the m/z range between 180 and 2,000 with a given bin width. The range was limited to 0-2000 because there are very few MS/MS spectra contain peaks with m/z above 2,000. This range may be extended if a larger m/z range is needed. By default, a bin width of 0.1 was used, resulting in vector representations of 20,000 dimensions.

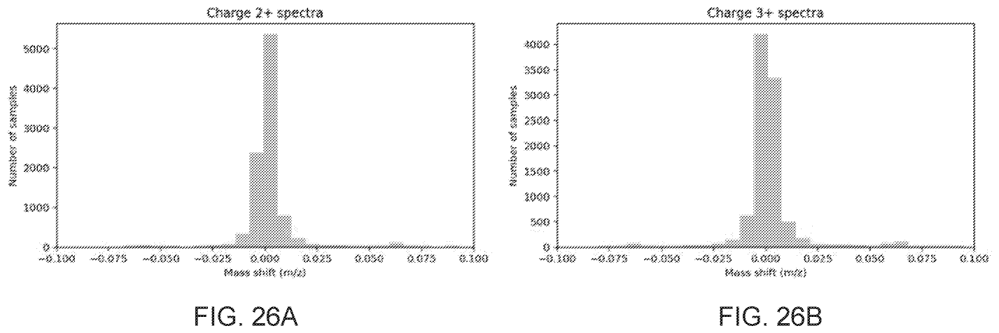

The default bin width was selected based on the observed m/z shifts between the corresponding peaks in replicated experimental spectra. As shown in FIG. 26, although many mass spectrum instruments often claimed a much higher mass precision, the observed m/z shifts are not ignorable when the bin width is lower than 0.05 m/z. Since a meaningful bin width must be slightly higher, the default bin width was selected as 0.1 m/z. In fact, it was determined that a smaller bin width (i.e., higher mass resolution) such as m/z of 0.05 will not improve the performance but will require much longer training times.

Finally, as the absolute intensities in the MS/MS spectra are irrelevant, all spectra in training and testing sets are normalized by dividing the maximum peak intensity in each spectrum. It should be noted that, in the illustrative embodiment, the precursor peak in each spectrum was removed, although the precursor peak was relatively weak in most spectra.

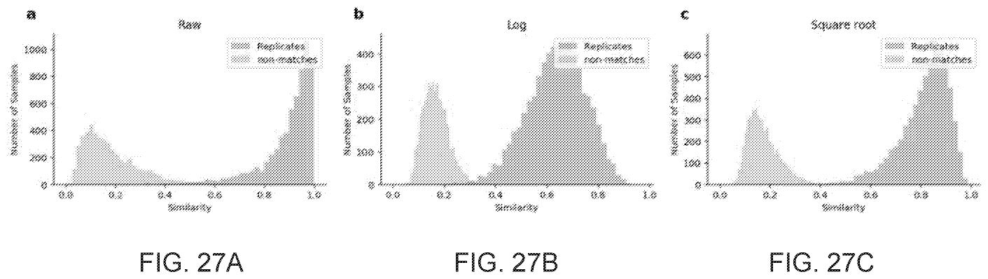

Evaluation Criteria and Intensity Transformation. Several metrics have been proposed to measure the similarity between two MS/MS spectra in the context of spectra identification and spectra library search. In the illustrative embodiment, the most widely accepted metric of cosine similarity (normalized dot product) between two spectra was selected as the evaluation standard. It should be appreciated that the similarities computed on unnormalized intensities are often misleading because the results may be dominated by a few very intense peaks in the spectra. As shown in FIG. 27A, when computing using the raw intensities, although the distribution of cosine similarities between replicated spectra are high, it is largely overlapped with the distribution of the similarities between the spectra of different peptides with similar precursor masses. In practice, several different transformation functions were suggested to reduce the impact of the most intense peaks when performing identification and comparison, such as logarithm or square root. In the illustrative embodiment, the square root function was selected for transforming peak intensities in each spectrum because the square root function exhibited similar effects as the logarithm function while negative values will not be introduced after the transformation. As shown in FIG. 27C, after the square root transformation, the similarity distribution of replicated spectra are better separated from that of the spectra from different peptides.

Prediction of Doubly and Triply Charged HCD Spectra. The illustrative experiments focused on the prediction of 2+ and 3+ HCD spectra of unmodified peptides, as a large number of identified 2+ and 3+ HCD spectra are publicly available. To do so, a convolutional neural network (CNN) using the Keras framework with Tensorflow back-end was implemented. In total, around 1.5 million 2+ spectra and 1 million 3+ spectra samples were collected for training, as shown in Table 3. For testing purposes, about 16,000 2+ spectra and 14,000 3+ spectra were held out from the peptides that do not overlap with the remaining training samples. Although the illustrative experiments focused on the prediction of MS/MS spectra from unmodified peptides, it should be appreciated that, in some embodiments, similar experiments may be used to predict modified peptides. It should be appreciated that when training the prediction model, types of instruments used to acquire the HCD spectra were not distinguished because the HCD spectra generated by different instruments (e.g., Orbitrap, Fusion, or Q Exactive) are highly similar. Since not all training data provide information about the normalized collision energy (NCE), all unlabeled data were assumed to have the NCE of 25%. However, it should be appreciated that the impact of NCE on the resulting MS/MS spectra is relatively small.

Architecture of the Convolutional Neural Network. Referring now to FIG. 12, a generalized sequence-to-sequence (Seq2Seq) model (also referred to as the prediction model in this application) was developed based on the structure of the residual convolutional neural network for predicting the full MS/MS spectra from peptide sequences. The input for the illustrative prediction model is a 27 by 23 matrix (up to 25 amino acid residues long) that contains the peptide sequence, the amino-acid masses, and other necessary meta-information. Specifically, row 1 to row 22 of the matrix are the one hot encoding of the input peptide sequence (including 20 amino acids, one ending character, and one padding character), while the last row contains the monoisotopic amino acid mass.

The embedded representation was first be fed into 8 parallel 1-dimensional convolutional layers of different kernel sizes (from 2 to 9). This step was designed to capture the correlations among subsequences of the input peptide. Afterward, the convolution results were merged into a single tensor, which is then passed through 10 consequential Squeeze-and-Excitation blocks, in the illustrative embodiment. However, it should be appreciated that, in some embodiments, a different number of consequential Squeeze-and-Excitation blocks may be used. Three subsequently residual blocks and the last 1-dimensional convolutional layer work as a decoder, which decodes the previous tensor into the final prediction vector of length 20,000 representing the final MS/MS spectrum. The default 20,000 length vector in the prediction model corresponds to the mass resolution of 0:1 m/z, as described above.

It should be appreciated that, in the illustrative embodiment, commonly used pooling layers were not incorporated in the architecture of the illustrative prediction model, except the last layer. Unexpectedly, not incorporating any commonly used pooling layers along with the residual convolutional network structure was determined to be critical for achieving a good performance according to the illustrative experiments. The entire prediction model contains about 19 million parameters and occupies a space of around 77 Mb, the details of implementation and training process is described below.

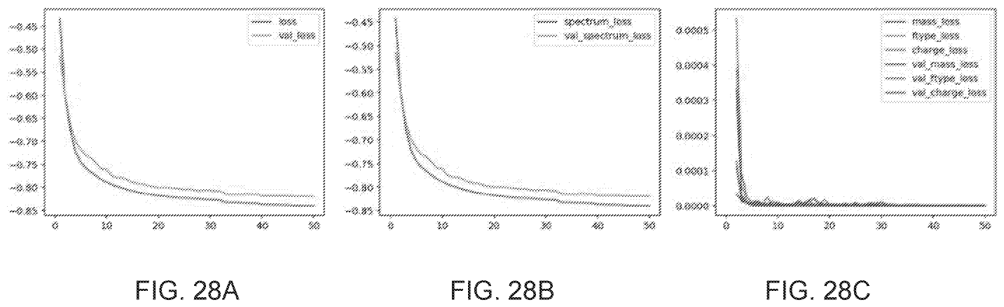

Implementation and Training. The CNN model was implemented in Python using the Keras framework with Tensorflow back-end. See, e.g., Chollet, F., et al. Keras. https://keras.io, 2015. A standalone software named PredFull was also implemented for predicting HCD spectra of given input peptide sequences. The software is released open-source on Github at https://github.com/lkytal/PredFull and can also be accessed through a web service at http://www.predfull.com/. The whole training and testing set was shared at http://www.predfull.com/datasets, including the raw experimental spectra, as well as the predicted spectra of the testing peptides in these datasets. The model was trained by Adam optimizer at a learning rate of 0.0003, with a batch size of 1024. See, e.g., Kingma, D. P.; Ba, J. Adam: A method for stochastic optimization. International Conference on Learning Representations (ICLR) (2015). The training process spans 50 epochs (FIG. 28), while the learning rate will be decay to 5×10⁻⁵ at the 30th epoch and 1.25×10⁻⁵ at the 40th epoch. The training process took around 12 hours (˜7×10⁻⁴ second per sample) using two NVIDIA GTX 1080ti GPUs, while the prediction takes ˜10⁻³ second per peptide.

Multitask Learning Framework.

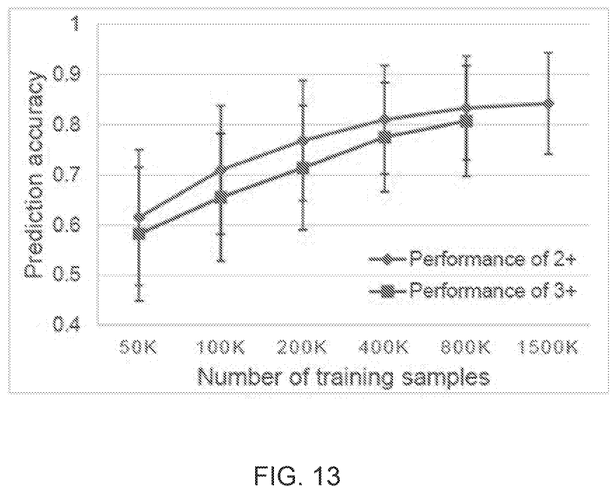

Prediction of 1+ and 4+ HCD Spectra with Insufficient Training Data. As stated above, around 2.2 million training samples were used for training the model to predict 2+ and 3+ HCD spectra. It is noted that the success of 2+ and 3+ HCD spectra prediction largely depends on the abundant training datasets. As shown in FIG. 13, the prediction accuracy increases significantly and steadily with more spectra are employed as training samples. However, the improvement of the performance started to gradually saturate when more than 1 million training samples were used. In the illustrative experiment, it was estimated that even with more training samples, the prediction accuracy of the illustrative prediction model may not further improve over 5%.

However, far less identified HCD spectra are available for the singly (1+) and quaternarily (4+) charged peptide ions. Thus, a multitask learning (MTL) approach that can train the illustrative prediction model with insufficient training samples was developed, which significantly improved the prediction accuracy when large training sets are not available. To do so, a universal model was implemented, which can be trained simultaneously by HCD spectra of different charges. This approach not only saves the efforts of building many models for different charges, but also improves the prediction performance, as the fragmentation mechanisms learned from charges with abundant spectra might also guide the prediction of charges with insufficient spectra.

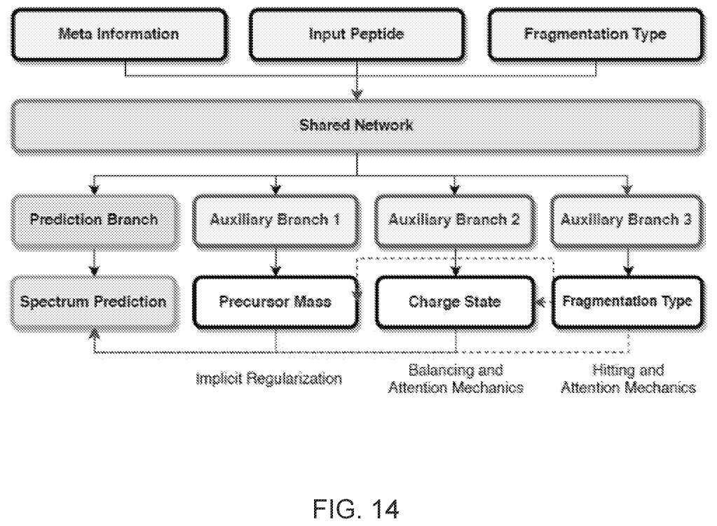

However, simply training a model by mixing all training samples together will not result in satisfactory performance because the neural network may easily be overwhelmed by the most abundant 2+ and 3+ spectra in the mixed dataset (known as “Catastrophic Forgetting”). Instead, auxiliary tasks may be used as a focusing method. Thus, the original prediction model was modified by adding an auxiliary task branch that “predicts” the precursor charges of the HCD spectra, as shown in FIG. 14. It should be noted that the illustrative experiments are not designed for predicting the charge state of the precursor since it is already given in the input. However, this prediction task informs the neural network with the importance of the desired charge state and enforces the prediction model to balance between the training samples of different charges. Additionally, an auxiliary task that “predicts” the precursor mass is given and also included). This auxiliary task works as a regulation to prevent overfitting and further stabilize the training process. As described further below, with the help of those auxiliary tasks, the illustrative universal prediction model significantly improved its performance on 1+ and 4+ HCD spectra, which confirmed that these tasks benefit from learning spectra of different charges together.

Prediction of ETD Spectra with Insufficient Training Data. Additionally, the illustrative experiments We are also interested in predicting the MS/MS spectra resulting from Electron-Transfer Dissociation (ETD). However, similar to 1+ and 4+ spectra, a number of collected identified ETD spectra were much lower compared to the HCD spectra. As shown in Table 3, around 180,000 identified ETD spectra were collected, which is less than 10% of the HCD training data). Specifically, the ETD PSMs are obtained by MSGF+ searching on the Kuster synthetic dataset with a mass tolerance of 40 ppm and limit the QValue (similar to FDR value) up to 0.002. Furthermore, this dataset is unbalanced, in which a majority (146,855 out of 191,454) are 3+ spectra. Thus, training directly using these samples probably will not provide a satisfactory performance.

As such, in the illustrative embodiment, the joint model was extended to predict both HCD and ETD spectra by adding one more auxiliary task that “predicts” the given information of the fragmentation type, as shown in FIG. 14. To ensure that the given fragmentation type will not be ignored, this auxiliary task is connected to all previous branches to allow the full network to be aware of the difference between different fragmentation types. As described further below, the prediction performance of ETD spectra was improved significantly by learning HCD spectra concurrently.

Running other Predictors. For pDeep, Github release (https://github.com/pFindStudio/pDeep/tree/master/pDeep2) was downloaded and executed for prediction, setting NCE to 30% and the instrument to QE. For the extended pDeep version, the Github release was re-implemented using Keras following the structure described by Zhou, X.-X.; Zeng, W.-F.; Chi, H.; Luo, C.; Liu, C.; Zhan, J.; He, S.-M.; Zhang, Z. pdeep: Predicting MS/MS spectra of peptides with deep learning. Analytical chemistry 2017, 89, 12690-12697, but extended the model to predict additional backbone ions (including a/x/c/y ions and their neutral loss derivatives) as well. Subsequently, the model was trained with the same training set as this work, using Adam optimizer at a learning rate of 0.0002. For Prosit, the Github source code was downloaded https://github.com/kusterlab/prosit for prediction. For DeepMass, the Github scripts was used to pre-process (https://github.com/verilylifesciences/deepmass/tree/master/prism) the input and the processed data was sent to their Google Cloud engine (as instructed in their Github pages) for spectrum prediction.

Results and Discussion for FIGS. 12-28:

Prediction Performance on 2+ and 3+ HCD Spectra of Peptides. To evaluate the accuracy of the predicted MS/MS spectra, the cosine similarities was computed between the experimental and the predicted spectra by the prediction model on the testing data of 16,000 2+ spectra and 14,000 3+ spectra, as shown in Table 3. For comparison, the similarities of predictions made by three best-performed models (i.e., pDeep, Prosit, and DeepMass) were computed. It should be noted that the similarities are much lower than those reported in their original publications because the similarities were computed with the complete experiment spectra and not with backbone ions solely. As discussed above, these models (i.e., pDeep, Prosit, and DeepMass) are limited to predict backbone ions. Furthermore, for each testing case, a theoretical perfect backbone spectrum consisting of only backbone ions from the experimental replicates was generated but removed all other ions. This represents the upper bound performance for all backbone only predictors.

As shown in FIGS. 15A and 15B, the spectra predicted by the illustrative prediction algorithm are highly similar with the experimental spectra. The average full spectrum cosine similarities (denoted as “This Work” in FIGS. 15A and 15B) were 0.820 (±0:088) for 2+ spectra and 0.786 (±0.085) for 3+ HCD spectra. This is very close to the average full spectrum cosine similarities between the replicated spectra of the same peptides, which were 0.837 (±0.114) for 2+ spectra and 0.806 (±0.113) for 3+ spectra, indicating that the illustrative prediction models approach the optimal prediction accuracy. In contrast, even the generated perfect backbone spectrum (denoted as “perfect backbone” in FIGS. 15A and 15B) only achieved the average cosine similarities around 0.750 (±0.124) and 0.700 (±0.127) for 2+ and 3+ spectra, respectively.

However, because it is impractical to achieve the perfect prediction in practice, the average cosine similarities achieved by the rule-based prediction were obtained by the extended implementation of pDeep (denoted as “full backbone” in FIGS. 15A and 15B) and were around 0.731 (±0.126) and 0.697 (±0.107) for 2+ and 3+ spectra, respectively. The original pDeep software as well as the more recently published software tools Prosit and DeepMass, which do not consider all possible backbone ions, can only achieve even lower average cosine similarities below 0.65, as shown in FIGS. 15A and 15B. As discussed above, the similarities listed above for pDeep, Prosit, and DeepMass are lower than those reported in previous studies because those previous results were calculated on only backbone ions but not on the full spectrum.

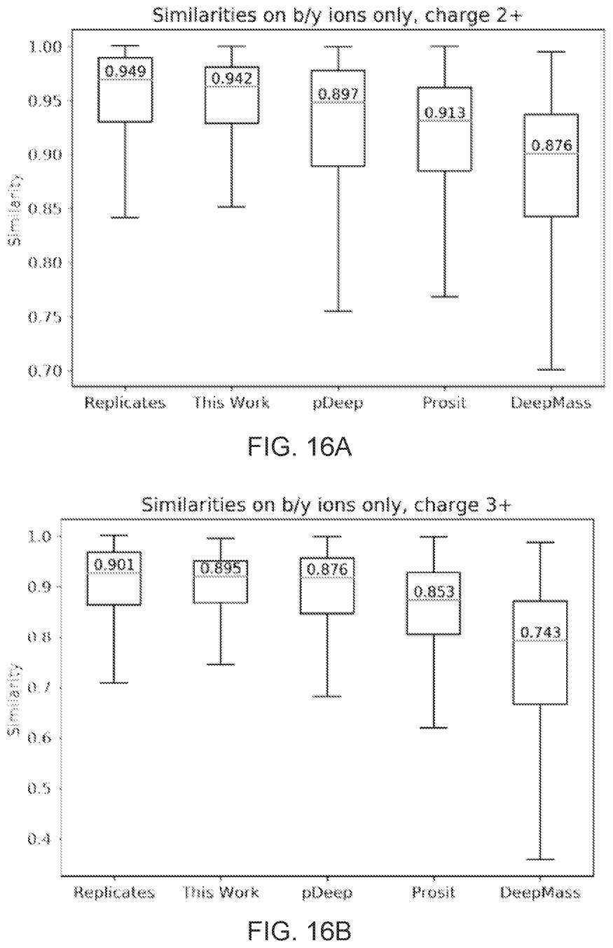

However, it should be appreciated that even in cases where only backbone ions were considered, the illustrative prediction model still outperforms all previous backbone only models. As shown in FIGS. 16A and 16B, the illustrative prediction model achieved highly accurate intensities prediction on b/y ions with average cosine similarities of 0.942(±0.075) and 0.895 (±0.070) for the 2+ and 3+ spectra, respectively, both approaching the similarity between replicated spectra and higher than previous backbone only models. This unexpected results indicates that the full-spectrum prediction benefits from learning and predicting all ions simultaneously. In other words, knowledge learned from non-backbone ions may also guide the predicting of backbone ions.

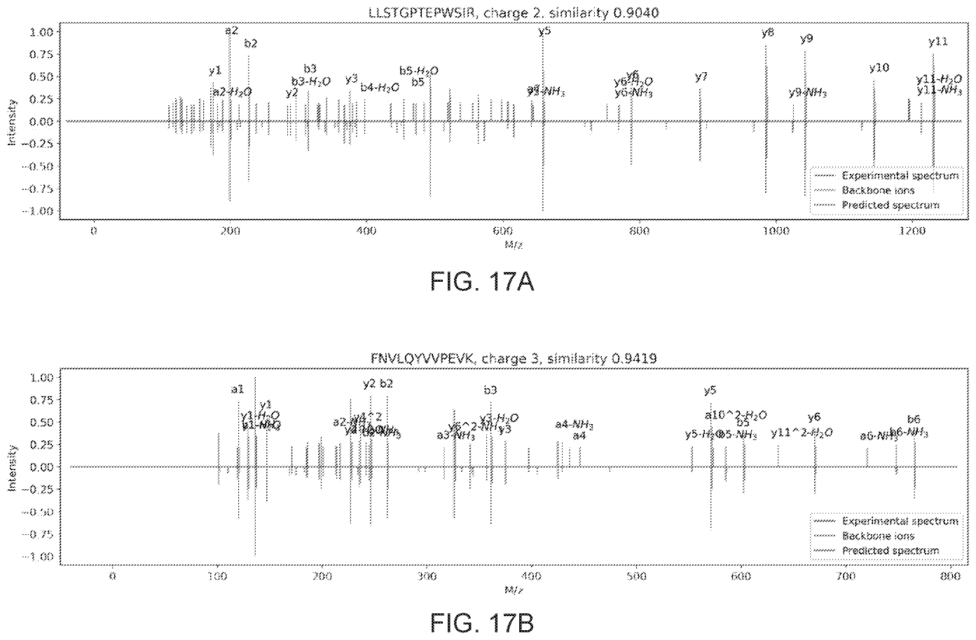

More specifically, as illustrated by two examples of prediction shown in FIGS. 17A and 17B, the illustrative prediction algorithm is capable of predicting the complete MS/MS spectra. In other words, the full-spectrum prediction model covered most intense non-backbone ion peaks observed in the experimental spectra, showing that these peaks represent fragmentation patterns that can be captured by the learning algorithm, even though the fragmentation mechanism remains unknown. Overall, the illustrative prediction algorithm demonstrated a clear improvement over previous prediction algorithms.

Furthermore, as shown in FIGS. 22A-D, the composition of fragment ions were compared in the predicted spectra versus experimental MS/MS spectra by depicting the average percentages of total intensities for different types of fragment ions. The composition of fragment ions in the predicted spectra by the illustrative prediction method is similar to that in the experimental spectra, confirming that the illustrative prediction algorithm can reliably predict non-backbone ions. In the experimental HCD spectra, ˜30% of total peak intensities are contributed by non-backbone ions, while for the predicted spectra it is ˜20%, which is smaller but still substantial. These predicted non-backbone ions significantly boosted the similarity of the predicted spectra. It should be noted that the overall non-backbone ion intensities in the predicted spectra are slightly lower than those in the experimental spectra, probably due to the presence of non-replicable noise peaks in the experimental spectra that are not predictable.

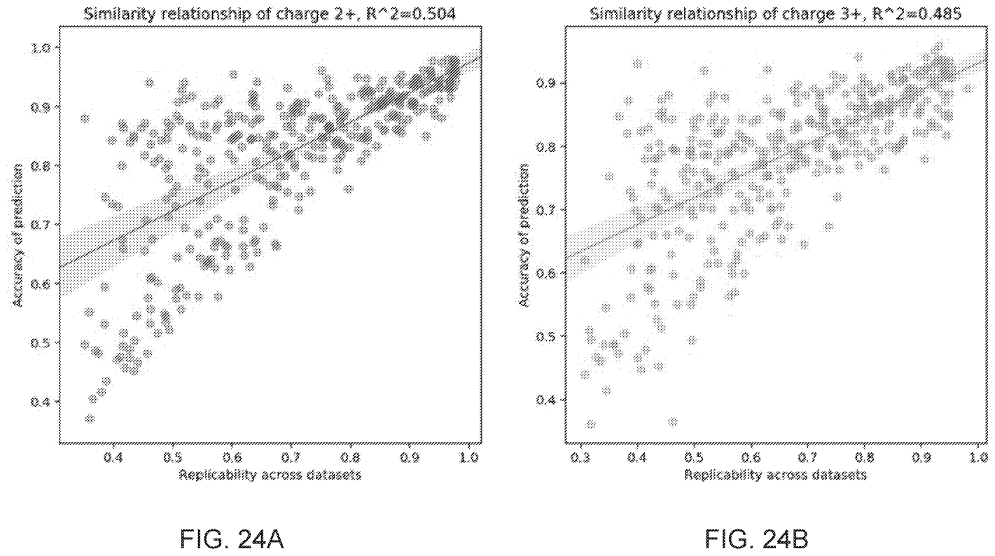

Variation of Prediction Accuracy. The replicated spectra of some peptides exhibited relatively low similarities. We investigated if the prediction similarities of these peptides are also relatively low. As shown in FIGS. 24A and 24B, the similarities between replicated HCD spectra are highly correlated with the similarities between the experimental and predicted spectra of the same peptide. This result confirms that the prediction performance largely depends on the replicability, while most of the poor predictions are caused by those less replicable peptides.

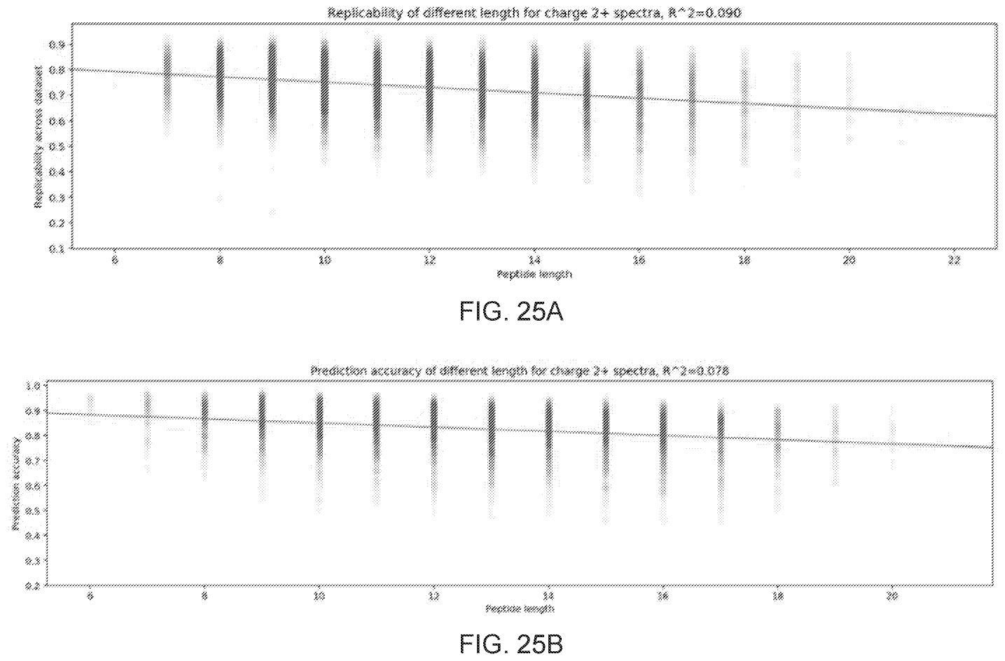

Additionally, the prediction accuracy of the illustrative prediction model varies depending on the peptide lengths and the replicability of the MS/MS spectra. As shown in FIG. 25A, the prediction accuracy decreases gradually with the increasing lengths of peptides, especially for peptides longer than 14 residues. Firstly, this may be because the spectra of long peptides may exhibit more complex fragmentation patterns, and thus made the prediction of long peptides more challenging. Secondly, the training dataset contains fewer samples of longer peptides, which makes it more difficult for the prediction model to learn the fragmentation rules and patterns for these peptides. Finally, the similarities between replicated experimental HCD spectra also decrease with the increasing peptide lengths as shown in FIG. 25B, indicating that the signal/noise ratio decreases in spectra of relatively longer peptides.

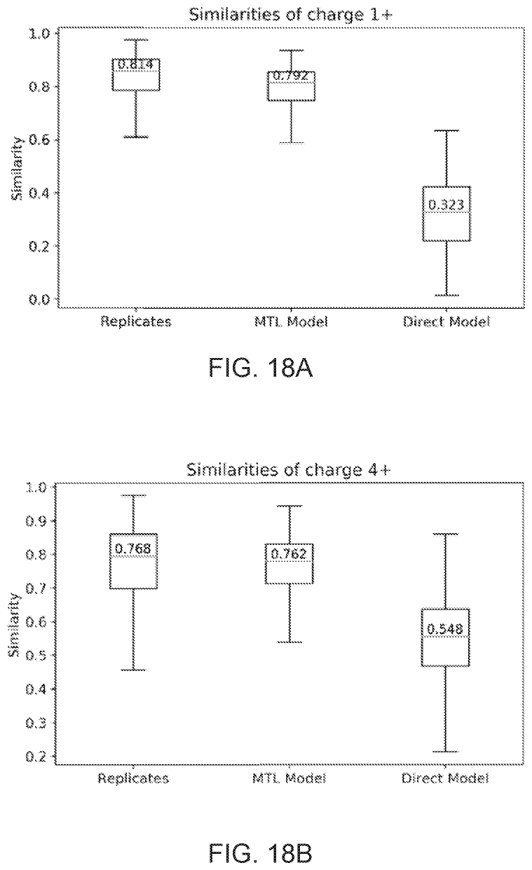

Prediction Performance on 1+ and 4+ HCD Spectra. The prediction performance of the illustrative multitask learning (MTL) model was evaluated using the training and testing datasets of 1+ and 4+ HCD spectra collected from the spectra libraries as described in Table 3. Because previous spectra prediction software (pDeep, DeepMass and Prosit) did not provide an option for predicting 1+ and 4+ spectra, the similarity between predicted and experimental spectra was compared with the experimental replication and the prediction model trained only using the training samples with the respective charges (e.g., the model for 4+ spectra prediction trained by using only 4+ spectra in the training set). As shown in FIGS. 18A and 18B, the MTL approach yields satisfactory performance with the similarities between the predicted and experimental spectra approaching that between the replicated spectra, which is much higher than those from the spectra prediction models trained directly from the subset of spectra with the specific charge (1+ or 4+).

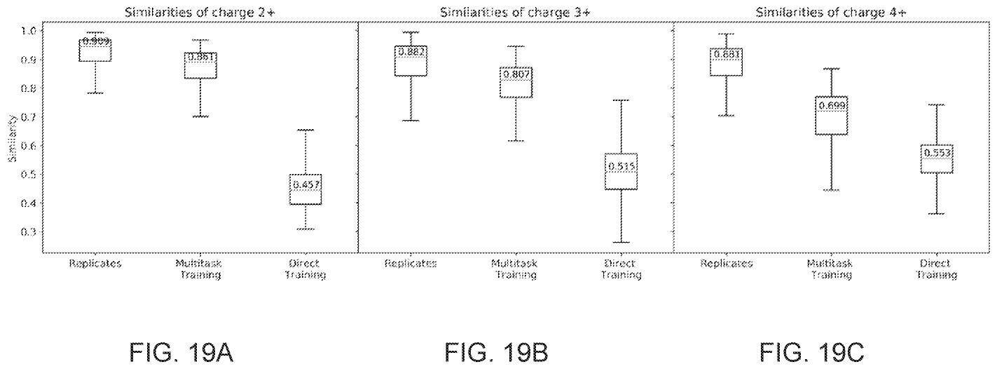

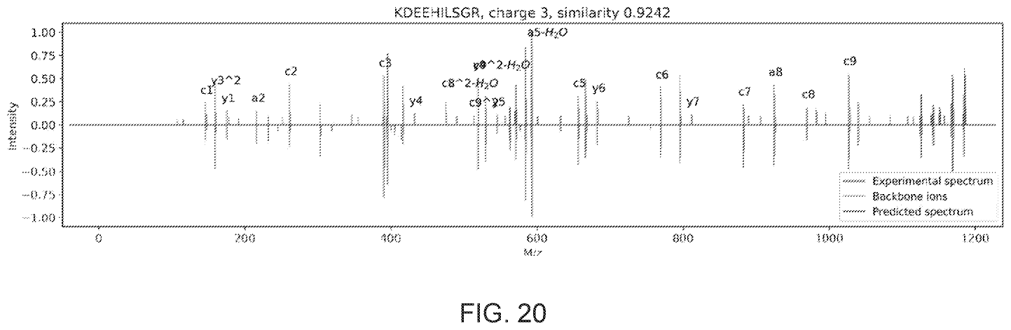

Prediction Performance on ETD. The prediction performance of the MTL model was evaluated using the training and testing datasets of ETD spectra collected from the spectra libraries as described in Table 3. Not surprisingly, without MTL approach, the average similarity between the experimental and predicted spectra is below 0.55 (denoted as “Direct Training” in FIGS. 19A-19C), far from the average similarity between replicated ETD spectra (e.g., ˜0.88 for 3+; FIGS. 19A-19C). However, by utilizing the joint MTL model, comparable average similarities were achieved using this relatively small ETD dataset (denoted as “Multitask Training” in FIGS. 19A-19C). An example prediction of ETD spectra is shown in FIG. 20.

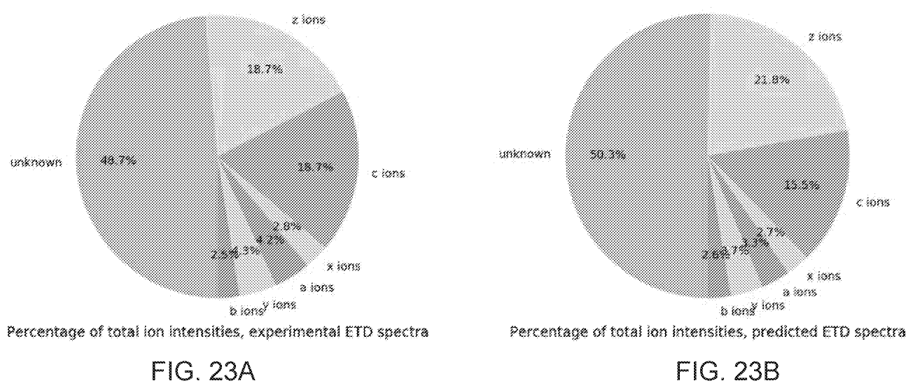

Interestingly, the intensity composition of the fragment ions in the predicted spectra is close to that of the experimental spectra. Like in HCD spectra, where b/y ions and their neutral loss derivatives comprise more than 60% intensities (shown in FIGS. 22A-22D), c/z ions are the most intense ions in ETD spectra (shown in FIGS. 23A and 23B). Notably, the fragmentation rules of these two methods (e.g., abundant b/y ions in HCD and abundant c/z ions in ETD) were not provided to the deep learning model; nonetheless, the illustrative prediction model discovered these patterns directly from the training data.

Conclusion for FIGS. 12-28:

The illustrative deep learning approach was presented for predicting the complete tandem mass spectra directly from peptide sequences without providing any prior knowledge. Such prediction model is different from existing backbone-only spectrum predictors (e.g., pDeep, Prosit and DeepMass), which are limited to predict only the intensity of an expected subset of fragment ions (i.e., backbone ions in HCD spectra). As described above, the illustrative prediction model predicts the non-backbone ions in HCD and ETD spectra, for which the fragmentation mechanisms may not be fully understood, leading to much higher overall prediction accuracy and ion coverage, as shown in FIGS. 15A and 15B. As discussed above, the multi-task learning (MTL) approach was also developed for training a joint prediction model, which significantly improved the prediction accuracy for spectra with insufficient training data (e.g., 1+ and 4+ HCD spectra and ETD spectra of all charges). The testing results showed that the model trained using the MTL approach achieved comparable performance on both types of tasks, with fewer than 200,000 samples were used for training.

It should be appreciated that, in some embodiments, the illustrative deep learning approaches may be extended to the prediction of MS/MS spectra using other fragmentation methods, e.g., the high energy HCD or electron transfer/high energy collision dissociation (EThcD), in which the fragmentation rules are more complex and less understood. In other embodiments, the illustrative prediction model may be extended for predicting spectra from modified peptides. Lastly, in some embodiments, other computational methods may be developed to automatically generate hypotheses about the explicit fragmentation mechanisms and/or rules resulting in the non-backbone ions with the help of complete spectra prediction.

Various modifications and additions can be made to the embodiments disclosed herein without departing from the scope of the disclosure. For example, while the embodiments described above refer to particular features, the scope of this disclosure also includes embodiments having different combinations of features and embodiments that do not include all of the described features. Thus, the scope of the present disclosure is intended to embrace all such alternatives, modifications, and variations as fall within the scope of the claims, together with all equivalents.

What is claimed is:

1. A method for predicting complete tandem mass spectra of a molecule, the method comprising:
training a prediction model using a dataset with features that incorporate at least one physiochemical feature derived from one or more peptide sequences; and
predicting complete tandem mass spectra of a molecule using the prediction model, the complete tandem mass spectra including backbone fragment ions and non-backbone fragment ions.

2. The method of claim 1, wherein the dataset includes a plurality of fragment peaks of the one or more peptide sequences without fragment ion annotations or fragmentation rules.

3. The method of claim 1, wherein training the prediction model using the dataset comprises:
inputting the at least one physiochemical feature derived from one or more peptide sequences; and
learning physiochemical rules governing peptide fragmentation to predict fragmentation rules.

4. The method of claim 1, wherein predicting MS/MS spectra of a molecule comprises predicting one or more occurrences and intensities of backbone fragment ions and/or non-backbone fragment ions.

5. The method of claim 1, wherein predicting the complete tandem mass spectra of the molecule comprises:
determining an intensity vector for each peak of experimental spectra and predicted spectra;
normalizing intensity vectors to avoid being dominated by one or more intensive peaks;
determining a cosine similarity of the normalized intensity vectors between experimental spectra and predicted spectra; and
comparing the cosine similarity.

6. The method of claim 1, wherein the molecule is selected from the group consisting of a peptide, a metabolite, a lipid, and a glycan.

7. The method of claim 1, wherein the molecule is a peptide.

8. The method of claim 1, wherein the peptide is a modified peptide.

9. The method of claim 1, wherein the dataset includes a plurality of fragment peaks from at least one of high-energy collisional dissociation (HCD) spectra, electron transfer dissociation (ETD) spectra, and/or collision-induced dissociation (CID) spectra.

10. A computing device for predicting complete tandem mass spectra of a molecule, the computing device comprising:
a processor; and
a memory having a plurality of instructions stored thereon that, when executed by the processor, causes the computing device to:
train a prediction model using a dataset with features that incorporate at least one physiochemical feature derived from one or more peptide sequences; and
predict complete tandem mass spectra of a molecule using the prediction model, the complete tandem mass spectra including backbone fragment ions and non-backbone fragment ions.

11. The computing device of claim 10, wherein the dataset includes a plurality of fragment peaks of the one or more peptide sequences without fragment ion annotations or fragmentation rules.

12. The computing device of claim 10, wherein to train the prediction model using the dataset comprises to:
input the at least one physiochemical feature derived from one or more peptide sequences; and
learn physiochemical rules governing peptide fragmentation to predict fragmentation rules.

13. The computing device of claim 10, wherein to predict MS/MS spectra of a molecule comprises to predict one or more occurrences and intensities of backbone fragment ions and/or non-backbone fragment ions.

14. The computing device of claim 10, wherein to predict the complete tandem mass spectra of the molecule comprises to:
determine an intensity vector for each peak of experimental spectra and predicted spectra;
normalize intensity vectors to avoid being dominated by one or more intensive peaks;
determine a cosine similarity of the normalized intensity vectors between experimental spectra and predicted spectra; and
compare the cosine similarity.

15. The computing device of claim 10, wherein the molecule is selected from the group consisting of a peptide, a metabolite, a lipid, and a glycan.

16. The computing device of claim 10, wherein the molecule is a peptide.

17. The computing device of claim 10, wherein the peptide is a modified peptide.

18. The computing device of claim 10, wherein the dataset includes a plurality of fragment peaks from at least one of high-energy collisional dissociation (HCD) spectra, electron transfer dissociation (ETD) spectra, and/or collision-induced dissociation (CID) spectra.

19. A non-transitory computer-readable medium storing instructions for a status of a mobile device of a user, the instructions when executed by one or more processors of a computing device, cause the computing device to:
train a prediction model using a dataset with features that incorporate at least one physiochemical feature derived from one or more peptide sequences; and
predict complete tandem mass spectra of a molecule using the prediction model, the complete tandem mass spectra including backbone fragment ions and non-backbone fragment ions.

20. The non-transitory computer-readable medium of claim 19, wherein the dataset includes a plurality of fragment peaks of the one or more peptide sequences without fragment ion annotations or fragmentation rules.

21. The non-transitory computer-readable medium of claim 19, wherein to train the prediction model using the dataset comprises to:
input the at least one physiochemical feature derived from one or more peptide sequences; and
learn physiochemical rules governing peptide fragmentation to predict fragmentation rules.

22. The non-transitory computer-readable medium of claim 19, wherein to predict MS/MS spectra of a molecule comprises to predict one or more occurrences and intensities of backbone fragment ions and/or non-backbone fragment ions.

23. The non-transitory computer-readable medium of claim 19, wherein to predict the complete tandem mass spectra of the molecule comprises to:
determine an intensity vector for each peak of experimental spectra and predicted spectra;
normalize intensity vectors to avoid being dominated by one or more intensive peaks;
determine a cosine similarity of the normalized intensity vectors between experimental spectra and predicted spectra; and
compare the cosine similarity.

24. The non-transitory computer-readable medium of claim 19, wherein the molecule is selected from the group consisting of a peptide, a metabolite, a lipid, and a glycan.

25. The non-transitory computer-readable medium of claim 19, wherein the molecule is a peptide.

26. The non-transitory computer-readable medium of claim 19, wherein the peptide is a modified peptide.

27. The non-transitory computer-readable medium of claim 19, wherein the dataset includes a plurality of fragment peaks from at least one of high-energy collisional dissociation (HCD) spectra, electron transfer dissociation (ETD) spectra, and/or collision-induced dissociation (CID) spectra.
